(19) United States
(12) Patent Application Publication
(10) Pub. No.: US 2011/0054266 A1
(43) Pub. Date: Mar. 3, 2011

(54) METHOD FOR PROTOCOL CREATION IN A DIAGNOSTIC IMAGING SYSTEM

(75) Inventors: Patrick Dinino, Hudson, NH (US); Robert Springer, Narberth, PA (US); Susan Heller, Lake Stevens, WA (US); Mary Lalomia, Snoqualmie, WA (US); Joseph M. Luszcz, Hudson, NH (US); Kris Bartol, Nashau, NH (US)

(73) Assignee: KONINKLIJKE PHILIPS ELECTRONICS N.V., Eindhoven (NL)

(21) Appl. No.: 12/808,715

(22) PCT Filed: Dec. 9, 2008
(86) PCT No.: PCT/IB2008/055171

§ 371 Date: Oct. 22, 2010

Related U.S. Application Data
(60) Provisional application No. 61/014,095, filed on Dec. 17, 2007.

(51) Int. Cl. A61B 5/00

(52) U.S. Cl. 600/300

(57) ABSTRACT

A method of exam protocol creation in a diagnostic imaging system includes initiating a non-protocol diagnostic imaging exam with the diagnostic imaging system. Concurrently with implementation of the initiated non-protocol diagnostic imaging exam, procedural characteristics of the non-protocol diagnostic imaging exam are recorded, wherein recording includes automatic system recording. In addition, the method includes defining a diagnostic imaging exam protocol in response to the recorded procedural characteristics of the non-protocol diagnostic imaging exam. Defining includes producing an automatic system generated definition of the diagnostic imaging exam protocol as a function of information extracted from the recorded procedural characteristics.

The present disclosure relates generally to medical systems and more particularly, to a method for protocol creation in a diagnostic imaging system.

Exam protocols, in general, are a common concept. Hospitals, ultrasound labs and even individual physicians all have specific, and often different, criteria which they consider essential in the process of conducting an ultrasound exam. The protocol itself is a list of the criteria that needs to be investigated during the course of an exam. The criteria can be broken down into small sections, each of which tends to have its own identification, notes about its origin and any results or measurements that may be necessary to report. In addition, an exam protocol is a recipe for acquiring all the images in a medical exam together with characteristics, such as imaging mode and settings, and/or diagnostic actions associated with each image, such as annotation, placement of body marker graphics, named measurements, and so on. Protocols are created for various exam types, and are used during acquisition of the exam to guide the technologist as the images of the exam are acquired, annotated, and measured.

Diagnostic imaging systems may be constructed with a number of “built-in” or “factory-defined” protocols for common study types, but as mentioned above, ultrasound labs, imaging organizations, or individual physicians usually have unique needs or personal preferences that are best served through the customer's creation of “custom protocols”. The process of creating a custom protocol is known to be tedious and thus presents a barrier to the use of protocols. The nonuse of protocols thus reduces an efficiency of the ultrasound lab. The known process of creating a custom protocol also requires a level of knowledge about the ultrasound system that is related to a corresponding system setup and not a simple operation.

Accordingly, an improved method for overcoming the problems in the art is desired.

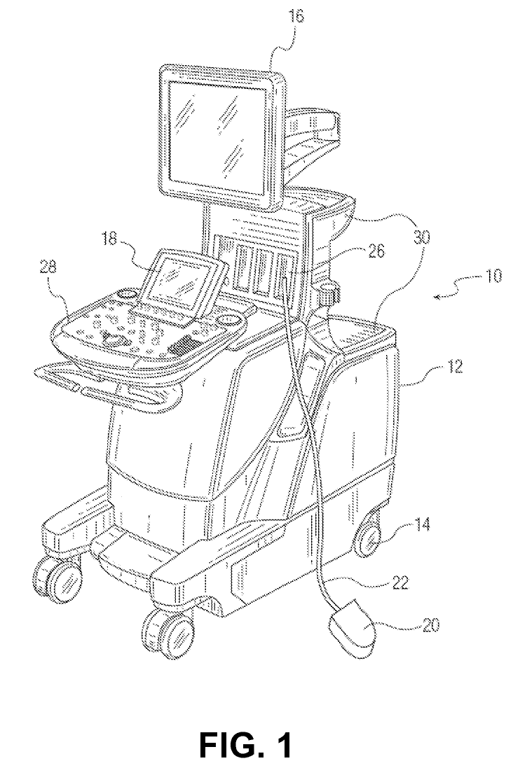

FIG. 1 is an isometric view of an ultrasound imaging system according to an aspect of the present disclosure;

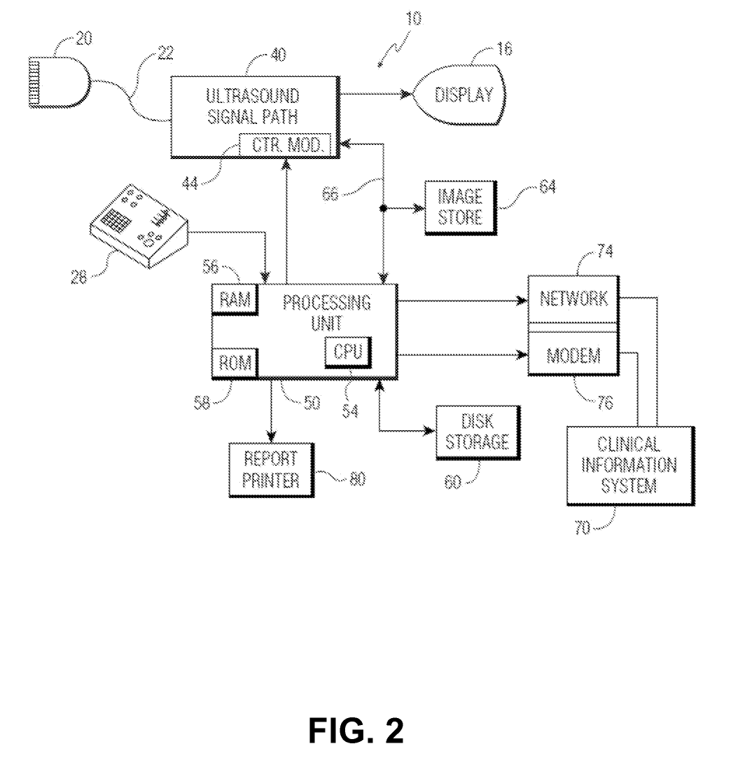

FIG. 2 is a block diagram view of the electrical components used in the ultrasound imaging system of FIG. 1 according to an aspect of the present disclosure;

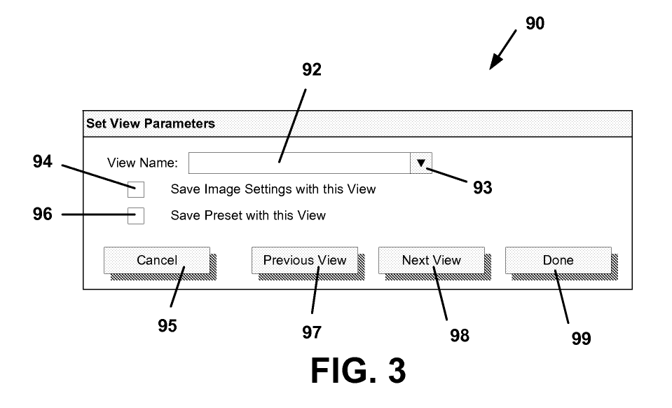

FIG. 3 is a dialog box view of a set view parameters feature of the diagnostic imaging system according to one aspect of the present disclosure;

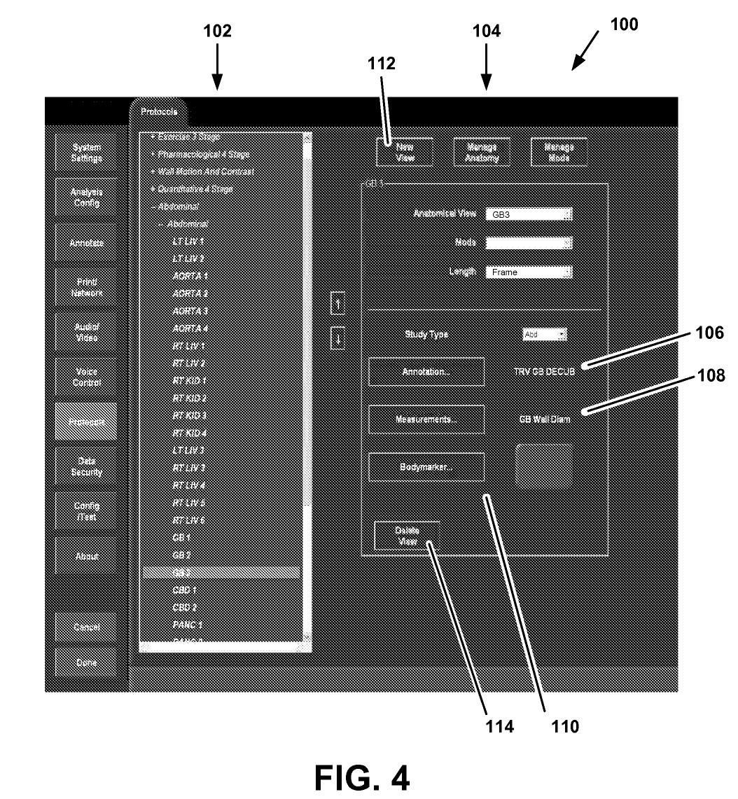

FIGS. 4-6 include examples of a monitor display view in the method for protocol creation with a diagnostic imaging system according to one aspect of the present disclosure;

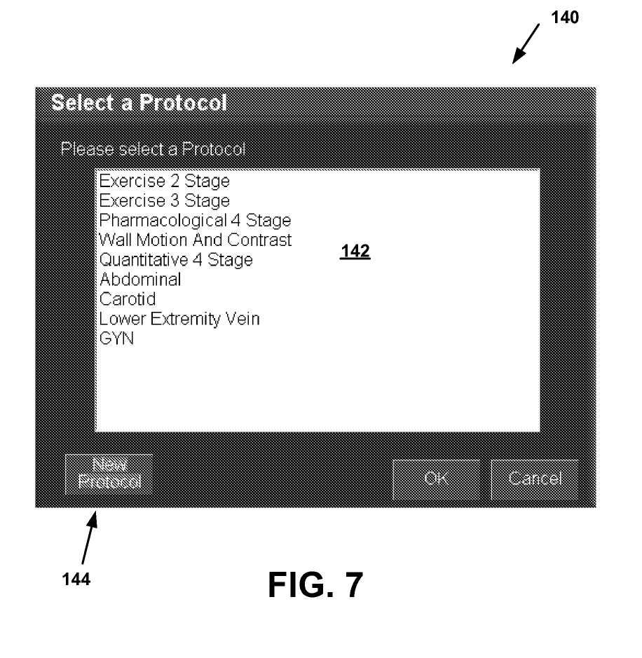

FIG. 7 is a dialog box view of a feature for selecting a protocol of diagnostic imaging system according to one aspect of the present disclosure;

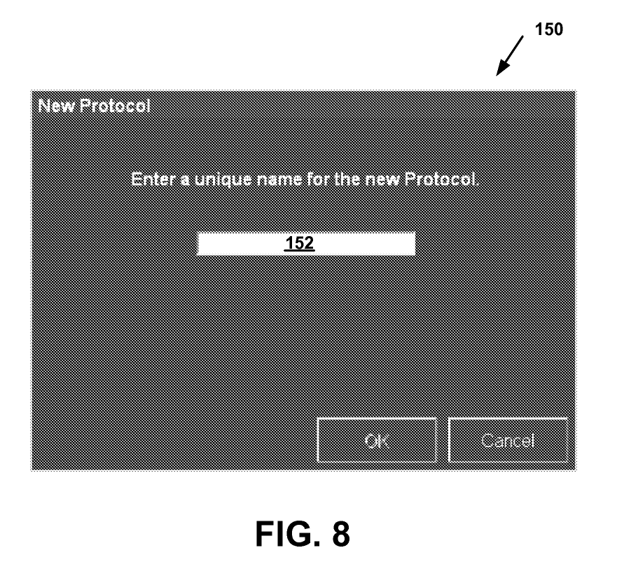

FIG. 8 is a dialog box view for naming of a new protocol of diagnostic imaging system according to one aspect of the present disclosure;

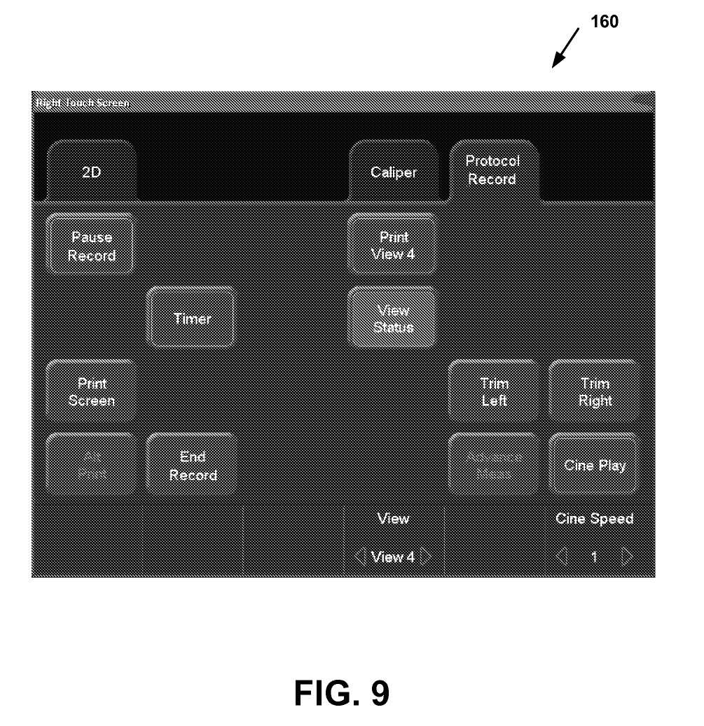

FIG. 9 is a diagrammatic view of a touch screen with various functionalities for protocol creation with the diagnostic imaging system according to one aspect of the present disclosure;

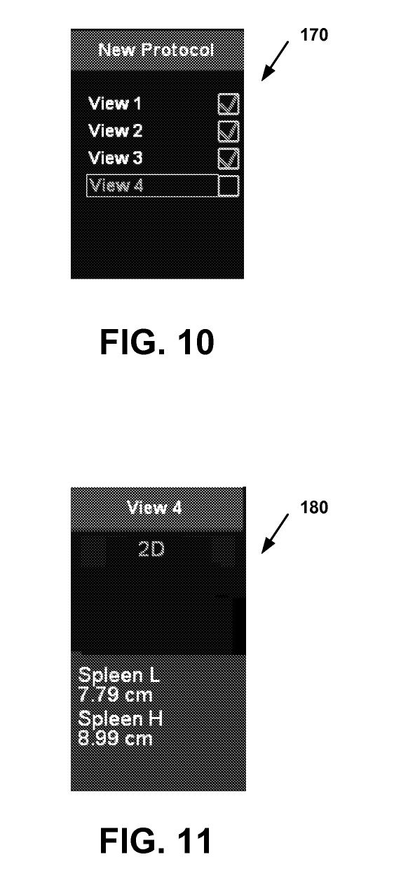

FIG. 10 is a diagrammatic view of one example of a protocol label region view list for protocol creation with the diagnostic imaging system according to one aspect of the present disclosure;

FIG. 11 is a diagrammatic view of one example of a protocol label region active view region for protocol creation with the diagnostic imaging system according to one aspect of the present disclosure; and

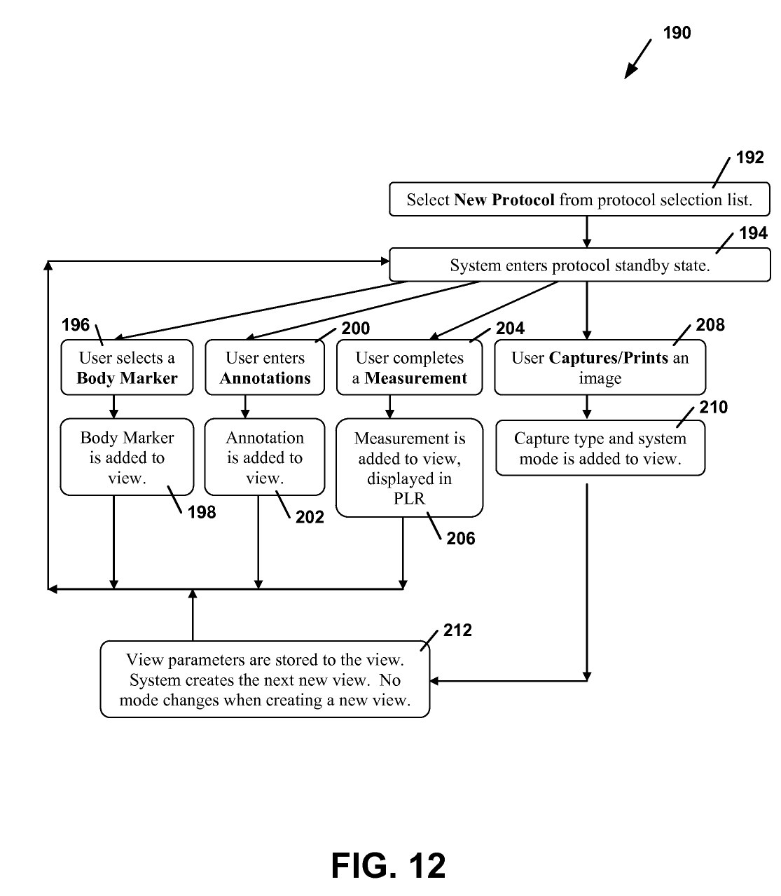

FIG. 12 is a flow diagram view illustrating a method for protocol creation with a diagnostic imaging system according to another aspect of the present disclosure.

In the figures, like reference numerals refer to like elements. In addition, it is to be noted that the figures may not be drawn to scale.

An ultrasound imaging system 10 according to one aspect of the present disclosure is illustrated FIG. 1. The system 10 includes a chassis 12 containing most of the electronic circuitry for the system 10. The chassis 12 is mounted on a cart 14, and a display 16 is mounted on the chassis 12. An imaging probe 20 is connected through a cable 22 to one of three connectors 26 on the chassis 12. The chassis 12 includes a keyboard and controls, generally indicated by reference numeral 28, for allowing a sonographer to operate the ultrasound imaging system 10 and enter information about the patient or the type of examination that is being conducted. At the back of the control panel 28 is a touchscreen display 18 on which programmable softkeys are displayed for supplementing the keyboard and controls 28 in controlling the operation of the system 10.

In operation, the imaging probe 20 is placed against the skin of a patient (not shown) and held stationary to acquire an image of blood or tissues in a volumetric region beneath the skin. A planar or volumetric image is presented on the display 16, and the image may be recorded by a recorder (not shown) placed on one of the two accessory shelves 30. The system 10 may also record or print a report containing text and images. Data corresponding to the image may also be downloaded through a suitable data link, such as the Internet or a local area network. In addition to using the probe 20 to show an image on the display, the ultrasound imaging system may also provide other types of information useful for a diagnosis, and it may accept other types of probes (not shown) to provide other types of images.

The electrical components of the ultrasound imaging system 10 are illustrated in FIG. 2. As mentioned above, the ultrasound imaging probe 20 is coupled by the cable 22 to one of the connectors 26, which are connected to an ultrasound signal path 40 of conventional design. As is well-known in the art, the ultrasound signal path 40 includes a transmitter (not shown) coupling electrical signals to the probe 20, an acquisition unit (not shown) that receives electrical signals from the probe 20 corresponding to ultrasound echoes, a signal processing unit (not shown) that processes the signals from the acquisition unit to perform a variety of functions such as isolating returns from specific depths or isolating returns from blood flowing through vessels and a scan converter (not shown) that converts the signals from the signal processing unit so that they are suitable for use by the display 16. The processing unit in this example is capable of processing both B mode (structural) and Doppler signals for the production of various B mode and Doppler images, including spectral Doppler images. The ultrasound signal path 40 also includes a control module 44 that interfaces with a processing unit 50 to control the operation of the above-described units. The ultrasound signal path 40 may, of course, contain components in addition to those described above, and in suitable instances, some of the components described above may be omitted.

The processing unit 50 contains a number of components, including a central processor unit (“CPU”) 54, random access memory (“RAM”) 56, and read only memory (“ROM”) 58, to name a few. As is well-known in the art, the ROM 58 stores a program of instructions that are executed by the CPU 54, as well as initialization data for use by the CPU 54. The RAM 56 provides temporary storage of data and instructions for use by the CPU 54. The processing unit 50 interfaces with a mass storage device such as a disk drive 60 for permanent storage of data, such as data corresponding to ultrasound images obtained by the system 10. However, such image data is initially stored in an image storage device 64 that is coupled to a signal path 66 extending between the ultrasound signal path 40 and the processing unit 50. The disk drive 60 also preferably stores protocols which may be called up and initiated to guide the sonographer through various ultrasound exams.

The processing unit 50 also interfaces with the keyboard and controls 28. The keyboard and controls 28 may also be manipulated by the sonographer to cause the ultrasound imaging system 10 to produce automatically generated reports at the conclusion of an examination. The processing unit 50 preferably interfaces with a report printer 80 that prints reports containing text and one or more images. The type of reports provided by the printer 80 depends on the type of ultrasound examination that was conducted by the execution of a specific protocol. Finally, as mentioned above, data corresponding to the image may be downloaded through a suitable data link, such as a network 74 or a modem 76, to a clinical information system 70 or other device.

According to one aspect, the diagnostic imaging system is provided with a protocol record feature, wherein a regular user familiar with simple operation of the system can create a protocol by executing an exam as they would outside of a protocol, while the diagnostic imaging system concurrently records a corresponding protocol for the exam. The protocol record feature advantageously saves the user the time of defining a protocol, and provides future exams the option of using the recorded protocol and realizing the benefits provided thereby.

In the aspect of the immediately preceding paragraph, the diagnostic imaging system featuring protocol record includes: a means for user initiation of the recording of an ultrasound exam; a means, responsive to the recording initiation, for the ultrasound system to record all keystrokes of the exam, extracting the elements of the exam that are needed to provide a definition for a future protocol; and a means for replaying the recorded protocol for use with future exams, as is discussed further herein.

It is noted that the Protocols feature of the known ultrasound system has enabled increased efficiency and effectiveness of ultrasound exams as compared to running the same exam on the same system without using a protocol. However, by administering a non-protocol exam, recording the corresponding exam and having the diagnostic imaging system create a protocol out of the recorded information according to one aspect of the present disclosure, a user now has the ability to run the corresponding created protocol the next time a similar exam is needed and realize the increase in efficiency and effectiveness of the system, not to mention providing a means to verify the exam is complete based on the protocol definition.

A pre-defined exam protocol for the known ultrasound system can include a given number of views or unique parts of an ultrasound exam that consist of an image, notes about the image, and measurements associated with the image. The image itself can come in two forms, a loop of data equivalent to a movie snippet and a still image/photo of the display. The exam itself can also span many different imaging modes on the system (2D, Color, 3D, etc). The system also provides various keys for a user to manipulate the system as necessary to complete the exam.

When defining an exam protocol on a known ultrasound system, the user must define all the views to be associated with the protocol being defined, as well as, all the steps to the exam, prior to actually carrying out an exam. This includes giving each view a name defining a system inode (2D, color, etc) that the system should be set to, defining the type of image the exam requires, adding notes about the image (if necessary), adding measurements to be taken on the image (if necessary), and adding body markers to be displayed during the acquisition of the view (if necessary). However, with the diagnostic imaging system of the present disclosure, as a user performs a non-protocol exam, a protocol recorder actively monitors for an occurrence of each of these actions and when one occurs, will store the corresponding action into a protocol definition. When a user has completed an image and all associated elements of that image, the user presses a button on the system that informs the protocol recorder that the current view is complete and that the user will now be starting a new view or, alternatively, ending the exam.

According to one aspect, the protocol recorder is configured to present, for example, on the system display, a current status of the new protocol being generated. The recorder assigns predefined names to the views as they are created and displays the corresponding names in sequential order on the display, wherein the recorder further provides the ability for a user to change the names if necessary. The recorder further highlights the active view being created, and displays all elements of the view (e.g., image type, system mode, etc) that have been defined based on the keystrokes or other inputs of the user to the system. The recorder also provides a user controllable means for deleting views already created during the exam. The recorder still further provides a user controlled means for verifying how a completed view has been defined. Furthermore, the recorder provides a user controllable means for ending the recording and verifying that all aspects of the newly created protocol are correct.

According to another aspect, a diagnostic imaging system with a protocol exam recording feature comprises means for recording keystrokes and operator usage of the system during an active exam for the purpose of packaging the system operations into a protocol that a user can ‘play back’ for future exams on a number of patients. Subsequent execution of the recorded protocol advantageously provides automated actions for the system operator, and thereby increasing efficiency and effectiveness of the corresponding exam.

According to another aspect, the diagnostic imaging system further comprises means for creating a protocol wherein the creating means includes protocol creation after-the-fact from a previously acquired clinical exam. In particular, the characteristics of each image in the exam, including the imaging mode, annotation, body markers, measurements taken, and so on, are stored in the created protocol. The user may then review the exam and add supplemental information to each image view, such as view name or anatomical codes, while looking at the image of that view. Accordingly, the protocol creation process is advantageously simplified.

The aspect advantageously eliminates the tedium of protocol creation by using an existing clinical exam as a “template” for creation of a protocol. An existing exam containing images acquired in particular imaging modes with particular annotations, body markers, measurements, etc. become the template for the newly created protocol.

The aspect of the preceeding paragraph provides a number of advantageous features as follows. With respect to a first feature, as an exam is acquired, the characteristics of each acquired image are remembered with the storage of the image, including imaging characteristics (e.g., mode, number of frames/beats/seconds, key settings, etc. and diagnostic decorations (e.g. annotation, body markers, measurements taken, and so on). With respect to a second feature, after the exam is completed, the full exam (or a portion of the exam) may be selected for review and the directive given to make this exam for portion of the exam) a newly created protocol, and a protocol name and/or coded name is assigned. With respect to a third feature, each image in the exam may then be viewed and a view name and/or coded view name added to each image. This information becomes part of the newly created protocol.

In addition, the aspects may also be provided with further functional features as follows. Edits may be made to a created protocol by “drag and drop” of one or more images from another previously acquired exam. Edits may also be made to a created protocol by “draw, and drop” of one or more views from another existing protocol. Edits may be made to a protocol during the course of the corresponding protocol exam acquisition by giving directive on a view of the protocol to add new images to the protocol, etc.

Implementation of the method according to the aspects of the present disclosure includes adapting the diagnostic imaging system to acquire each image as “rich data”. The “rich data” preferably includes at least enough information sufficient for creating a protocol from an image set after-the-fact. The aspect is further configured for ensuring the “rich data” definition is complete with all characteristics required for a given protocol definition, and the addition of user interface (UI) functionality for specifying: that an exam is to be made into a protocol, a dialog box to allow the user to specify a protocol name or protocol code sequence, and UI mechanisms to allow the user to specify a view name or view code sequence for each view in the exam. Additional software and Ulf functionality could be added to permit modification to the exam in-use, drag and drop changes to the protocol from review of another exam, drag and drop changes to the protocol from views in another protocol, and so on.

In one aspect, the diagnostic imaging system includes means for creating a custom protocol from an existing exam, i.e., a previously acquired exam. The diagnostic imaging system is adapted to record or capture “rich data” as part of the original exam. The ultrasound imaging system is further configured to acquire images and store “rich data.” in other words, acquired images are stored together with a significant amount of ancillary information pertaining to the acquired image. This supplemental information provides contextual information from which the protocol definition may be extracted. The supplemental information can include, for example, imaging mode settings, text and placement of screen annotation, symbol and placement of a “body marker” graphic, measurements acquired with the image, and so forth. The collection of this supplemental information in the natural flow of events makes it possible to create a protocol definition after-the-fact, lacking only the labeling of the images as “views” in the protocol when the user decides to create the protocol definition from the prior exam. Accordingly, the system enables the creation and/or editing of a custom protocol from an existing exam.

In another aspect, a method of creating a custom protocol comprises using a previously acquired set of images as a template. The set of images can be obtained from a previously acquired exam or part of a current non-protocol exam. The set of images may also be acquired from more than one previously acquired exams or parts of exams. To obtain the set of previously acquired images, the method includes opening a source exam using an exam review command, selecting one or more image(s), and creating a new protocol while the images remain selected. In one aspect, a user right-clicks on one of the selected images and then chooses from a drop-down menu one of (i) “Create General Imaging (GI) Protocol” to create a new protocol or (ii) “Add to GI Protocol” to add these views to an existing protocol. Note that only images that have associated “rich data” may be used to create protocol views, including (i) all images acquired on the current system and not subsequently deleted, plus (ii) all images exported/imported with “rich data.” The above menu choices are “grayed out” (i.e., not selectable) if rich data isn't available in all selected images.

If “Create GI Protocol” is chosen, each image is displayed in turn (i.e., in succession or a given sequential order) with a Set View Properties dialog box 90, for example, as illustrated in FIG. 3. After entering a name 92 (and other desired settings 94 and 96) a button 98 is provided to move to a Next View. After all views are named, the user is asked to specify a Protocol Name for the new custom protocol and the protocol is stored under that name.

If “Add to GI Protocol” is chosen, a dialog box is displayed that asks the user to select (i) the protocol to which these views are being added, and (ii) the placement of these views among the existing views in the protocol. The new views are inserted as a block into the existing view sequence; if the user wishes to place individual views in different locations within the existing views, a program setups feature is used to move the views around individually. Once the views placements are entered, each added image is displayed in turn (i.e., in succession or a given sequential order) with the Set View Properties dialog box 90, as shown in FIG. 3. After entering a name (and other desired settings), a user selectable button is provided to enable moving from the current view to the next view in the sequence. After all views are named the user is asked where to save the changed protocol. If the source protocol is a custom (modifiable) protocol, the user has a choice of (i) saving the changes in the original protocol or (ii) specifying a name to give a new protocol. If the source protocol is a built-in system protocol, then the user is required by default to specify a name to store changes as a new custom protocol.

The “Set View Parameters” dialog box 90 allows the user to enter a name and other parameters for the corresponding view. Behavior of the Set View Parameters dialog box 90 is as follows:

View Name [combo box] 92: The user may enter a free-form textual name for the view, or may choose from the pull-down list 93 of View Names used in other presets. Save Image Settings with this View [checkbox] 94: Checking this box preserves all “gain save” imaging settings with this view. Default is unchecked.

Save Preset with this View [checkbox] 96: Checking this box saves the preset with this view, while leaving this box unchecked leaves the preset unchanged when entering this view. Default is checked.

Cancel [button] 95: Cancels the operation and removes the (partial) new protocol. If one or more views have already been named, the user is asked to confirm the cancel operation.

Previous View [button] 97: Saves the current view parameters and returns to the previous view, allowing the user to modify the view parameters previously set. This button is grayed out if the first view is the current view or if the View Name 92 is blank.

Next View [button] 98: Saves the current view parameters and moves to the next view. This button is grayed out if the last view is the current view or if the View Name 92 is blank.

Done [button] 99: Saves view parameters and brings up a “Setup Associated Presets” dialog: box with the initial list of associated presets equal to the set of all presets used by the new views in the protocol, combined with the associated presets of the protocol being added to, if adding to an existing GI Protocol. This button is grayed out if one or more views have not been named.

Creating a protocol according to the aspects of the present disclosure makes it possible also to retain positions of annotation and body markers as part of the view definitions. In addition, a feature setup provides for a method of creating and editing views. In another aspect, source images (de-identified) from which the protocol was defined can be retained as tutorial “sample images” to guide inexperienced users on the views to acquire.

According to yet another aspect, the diagnostic imaging system further comprises means for automating exam protocols. In particular, the diagnostic imaging system comprises an ultrasound system provided with means for guiding a sonographer through an exam. The guiding means of the ultrasound system prompts the sonographer on what image(s) are necessary to capture, how to annotate the image, and whether or not measurements are necessary for the image. The ultrasound system is further configured with a means for cross checking that all necessary parts of the exam have been completed. Still further, the ultrasound system comprises means for creating personal protocols, wherein a sonographer can create his or her own personal protocol(s).

The aspect of the preceding paragraph overcomes problems in the art as follows. In almost all cases, the execution of a section of a protocol involves annotating the display, making a measurement, labelling the result of the measurement, and capturing an image of the display for exam records. One of the most time consuming aspects of the exam is the overhead of documenting the images and general system preparation leading up to the image. Fortunately, almost all of the necessary steps to be taken in preparation are known beforehand, as dictated by the exam protocol. By pre-programming this information into the ultrasound system, the user is spared the time necessary to do this at exam time, allowing the user to focus on the actual exam, reduce exam time, increase throughput and also provide a means to verify all aspects of an exam are completed. In addition, the system is adapted to allow a user the ability to pre-program unique information tailored to their individual preferences, wherein the protocol can be further enhanced to make the exam more applicable and efficient to the user.

Features of diagnostic imaging system according to one aspect of the present disclosure include: automatic annotation display; automatic body marker display; automatic measurement launching and assignment; automatic system parameter and mode settings; and an ability to modify all the above settings. The aspect further includes a visual display of exam status, including complete and incomplete sections, along with results. The aspect still further includes automatic linkage of exam findings to final exam report conclusions. The various features of the aspect also provide a training means for sonographers unfamiliar with the current protocol exam.

The automatic annotation display feature decreases the time a sonographer spends entering details about the resultant image they are adding to the exam. The automatic body marker display feature decreases the time a sonographer spends manipulating the body marker image included on the resultant image they are adding to the exam. The automatic measurement launching and assignment feature decreases the time a sonographer spends creating measurements on the resultant image they are adding to the exam. The automatic system parameter and imaging mode settings feature decreases the time a sonographer spends adjusting the system to the proper settings to acquire the resultant image they are adding to the exam.

In addition, the ability of the diagnostic imaging system to modify all the above settings provides a means to customize a protocol and tailor it to the specific needs of the sonographer, patient, physician, or ultrasound lab. This versatility also provides a means for a protocol to conform to accreditation standards set by governing bodies, enabling the particular ultrasound lab applying for accreditation to follow a given set of standards.

Furthermore, visual display of exam status provides the sonographers a means to prepare for an upcoming image and crosscheck the completeness of the exam, verifying that all necessary images have been completed. Automatic linkage of exam findings provides a means for the sonographer to enter additional data pertaining to a particular finding, data that may be outside the protocol's scope (e.g., an abnormal pathology), to the exam's final report while the sonographer is currently engaged with the unique finding. The aspect also provides a training means for sonographers unfamiliar with the current protocol exam.

An example of a protocol is an abdominal protocol. The abdominal protocol can consist, for example, of thirty-one (31) “views” or images that a sonographer would be required to capture for the protocol exam. In one version of the protocol package, each view can have an annotation, body marker, and measurement associated with it.

Examples of several views included in the abdominal protocol are:
Name: LT LTV 1 (shortened for easy reading as it will be displayed on the display monitor)

### Actual Anatomy Name: Sagital Left Liver

### System Annotation: SAG LT LIVER

### Associated Measurements: None

### Associated Body Markers: None

Name: RT KID 1 (shortened for easy reading as it will be displayed on the display monitor)

### Actual Anatomy Name: Sagital Right Kidney

### System Annotation: SAG RT KIDNEY

### Associated Measurements: Right Kidney Length

### Associated Body Markers: None

Name: GB3 (shortened for easy reading as it will be displayed on the display monitor)

### Actual Anatomy Name: Transverse Gall Bladder Decub

### System Annotation: TRV GB DECUB

### Associated Measurements Gall Bladder Wall Diameter

### Associated Body Markers: None

The diagnostic imaging system is further configured to allow one or more “elements” of the view to be edited and modified in any way, the “elements” including but not limited to name, annotation, measurement and body marker. In one aspect, the number of views for a protocol is unlimited and the diagnostic imaging system is further configured to allow a user to add or remove views as necessary.

The display image 100 of FIG. 4 shows a partial definition of a protocol. The left side 102 includes view names (LT LW 1, LT LW 2, AORTA 1, etc.) and the right side 104 includes the specific elements associated with a particular view (in this case, GB 3). The annotation 106 is displayed (IRV GB DECUB) and the measurement 108 is also displayed (GB Wall Diam (i.e. wall diameter)). All elements can be modified using the buttons which are generally indicated by reference numeral 110 (e.g., ‘Annotation . . . ’, ‘Measurements . . . ’, ‘Bodymarker . . . ’). Views can be added to the protocol using the ‘New View’ button 112 and views can be removed from the protocol using the ‘Delete View’ button 114. Further enhancements to the aspect of FIG. 4 can include providing association of ultrasound system settings, including imaging modes (e.g., 2D, Color, 3D) to the views for automatic launch.

With respect to executing a protocol, a system user launches the predefined protocol via appropriate control on the diagnostic imaging system. The system reads the definition of the protocol and, starting at the first view, reads all the elements associated with the view. The system automatically displays any annotations associated with the view and prompts the user to capture the image associated with the given name of the view. The system will also prompt the user if a measurement is necessary for the given view.

The diagnostic imaging system determines when the user has completed the necessary steps to complete the current view and advances the protocol as necessary, indicating on the display the updated status of the protocol.

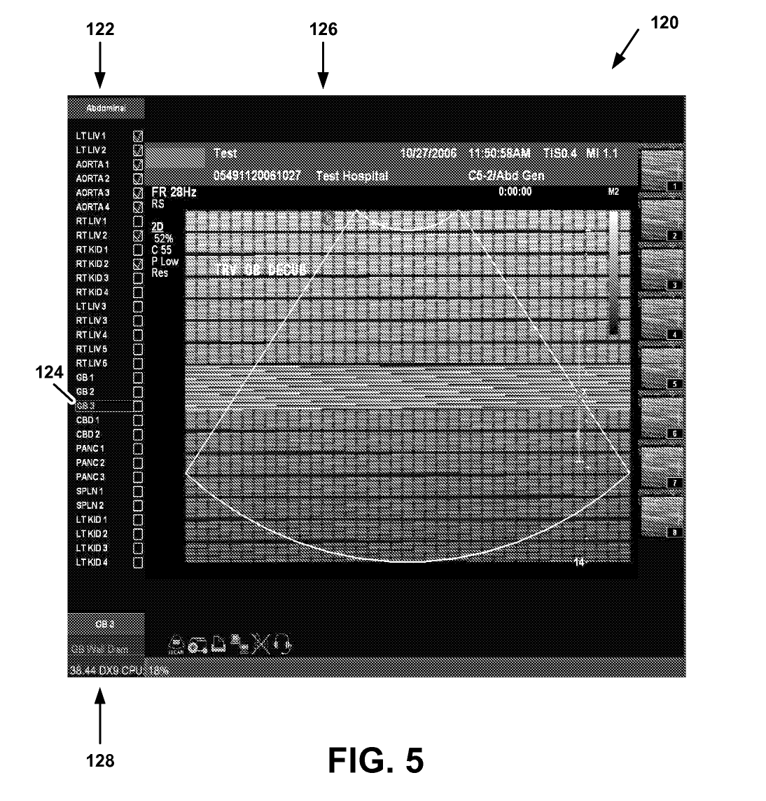

The display image 120 of FIG. 5 shows a protocol in progress. The left side 122 of the screen gives the status of the protocol, with a ‘checkmark’ next to names of views which have been completed. The currently active view (GB3) 124 is surrounded by a box, the annotation 126 for the active view is displayed on the screen (TRV GB DECUB), and the user is prompted that a measurement is associated with the view on the bottom left 128 of the display (i.e., GB Wall Dim).

When the protocol is completed, the system transitions to an ‘acceptance’ state where the user can review the exam and determine if any additional information is needed. The protocol also provides prompts to the user if parts of the protocol were determined to have not been completed, for example, as may have been required by the protocol.

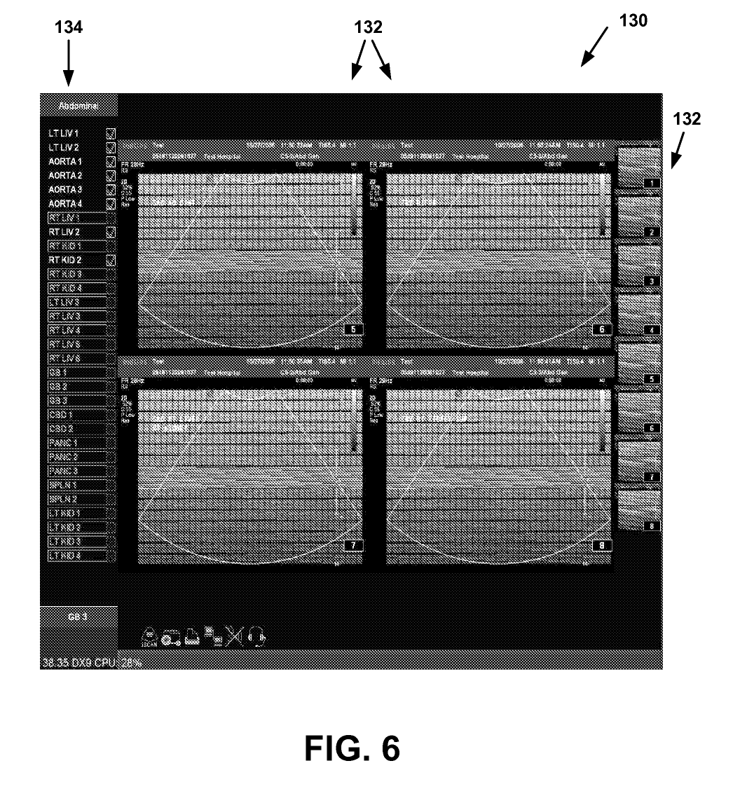

The display image 130 of FIG. 6 shows a ‘completed’ protocol exam. Captured images 132 are presented via the display to the user, along with a list 134 of the status of the protocol. Views that were completed have a check next to their corresponding name, and views that were not completed are highlighted by a box, followed by another box (for example, colored red) where a check mark would be if the same had been completed. FIG. 7 is a dialog box view 140 for selectin a protocol feature of diagnostic imaging system according to one aspect of the present disclosure. The dialog box 140 contains a listing of available protocols 142 from which a user can make a selection and then select the OK button to proceed with the selected protocol. For example, the medical diagnostic system includes a given number of default exam protocols in dialog box 140. The default exam protocols represent factory-defined protocols. Examples may include one or more of a. Abdominal, b. Carotid, c. Lower Extremity Venous, J. Gynecological (GYN), and other protocols. In addition, dialog box 140 includes a New Protocol button 144. In one aspect, selection of the New Protocol button 144 also serves as the user selectable key provided to initiate the protocol record feature as discussed herein. In other words, in one aspect, user selection of the New Protocol button 144 initiates a non-protocol diagnostic imaging exam and the concurrent recording of procedural characteristics of the non-protocol diagnostic imaging exam, wherein the recording includes an automatic system recording.

In accordance with one aspect of the present disclosure, the protocol record feature is designed to enhance and expedite the process of creating a user defined exam protocol. The process involves removing the necessity of entering setups, rather letting the user go through the actions of an actual exam, capturing prints, entering annotations, making measurements, etc. that are involved in completing a real exam. The diagnostic imaging system records the necessary steps taken during the non-protocol exam that are applicable to a user defined protocol definition and, from that information, creates a custom protocol.

The user interface (UI) of the diagnostic imaging system includes a Protocol Label Region (PLR), a status window for the protocol, and a layout for keys on a Protocol touch screen tab. In one aspect, a user selectable key is provided via the UI to initiate the protocol record feature.

According to another aspect, the diagnostic imaging system includes an automatic imaging mode switching (AIMS) feature which creates the ability for the user to define what imaging mode the system should be set to for different views in an exam protocol.

The system will automatically switch to the appropriate imaging mode when the active view is acquired.

In addition, the system provides a system user with the ability to enable and disable Automatic Imaging Mode Switching for an entire exam protocol. For a General Imaging Protocol (non-stress) the system is configured to assign an imaging mode which the system will switch to when the associated view becomes active. Examples of modes supported by the diagnostic imaging system can include: Color, Color/CW, and Color/PW, Echo2D, Echo2D/CW, Echo2D/M-Mode, Echo2D/PW, SonoCT, SonoCT/M-Mode, SonoCT/PW, CPA, CPA/CW, CPA/PW, Freehand 3D standby, and Motorized 3D standby. Furthermore, the system is configured to support various 3D and non-3D triodes. Examples of various non-3D modes can include one or more of: 2D; Dual; CW; M-Mode; PW; Color; Dual/Color; Color Compare; Color/CW; Color/M-Mode; Color/PW; CPA; Dual/CPA; CPA Compare, or other non-3D mode. Examples of various 3D modes can include one or more of: 3D Standby; 3D Sweep; 4D; STIC; 3D/Color Sweep; STIC/Color; 3D/CPA Sweep; STIC/CPA; or other 3D mode. Moreover, the system is configured to initiate a protocol record when selecting a new protocol.

FIG. 8 is a dialog box view 150 for naming of a new protocol feature of diagnostic imaging system according to one aspect of the present disclosure. In particular, the dialog box 150 includes a text box 152 for the user to enter a name for the new protocol. Upon entering the new protocol name, the user selects the OK button and the process continues. In one aspect, upon naming the new protocol via dialog box 150 and selecting the OK button, the system enters a protocol live state. The system automatically creates a single view for the protocol, calling it, for example, “View 1” and this view is considered active as a result of the protocol recorder having started. The new protocol displays one or more protocol touch screens which are available, for example, for running a given protocol or previously defined protocol.

The system creates a new view after each captured/printed image, placing the new view at the end of the protocol. This view will be known as the ‘Active Recording View’, or ARV. The ARV is defined as the view whose parameters have yet to be defined and is not currently included in the protocol. From the user's point of view, this will be the only view on the Protocol Label Region (PLR) that does not have a ‘check mark’ besides its name. Once the user initiates a capture or a print, this view will be recorded into the protocol and a new ARV will be created. If the system is configured for ‘Acceptance Prior to Store’ for loop captures, the user accepts the loop before the view will be considered complete.

While the ARV is the selected view, any annotations entered by the user, any body markers selected by the user, and any measurement completed by the user from the point the ARV was created will be associated with the ARV. The active imaging mode at the time of capture will also be associated with the view, the later requirement being dependent on requirements of a particular implementation of the Automatic Imaging Mode Switching feature. The state of the ARV is maintained if the user pauses the protocol, or uses the view knob to select a previously completed view. When the ARV becomes the selected view again, all previous information associated with the view will be recalled.

If the user navigates to a previously completed view, the lower portion of the PLR will show the detail of the selected view, and not of the ARV. In this given scenario, if the user captures any more images, or makes another measurement(s) and captures an image, the protocol recorder will immediately create a new view using the currently displayed annotations, body markers, imaging mode, and new measurements (if applicable). This new view is independent of the ARV and will be placed in the protocol immediately after the last completed view. If the user navigates back to the ARV, all previous settings for that view will be recalled. The protocol recorder proceeds in this fashion, creating new views and assigning view parameters, until either the user selects ‘End Exam’ or selects ‘End Protocol’.

In one aspect, the system places a limit on the number of views allowed per protocol. If, during a protocol record, the user reaches this limit, the system is configured to present a message indicating that the maximum view limit has been reached and the protocol record will end. The user will be able to complete their exam as needed, but no additional information will be saved into the protocol. In another aspect, the system places a limit on the number of measurements that can be associated with a view. If, during a protocol record, the user reaches this limit, the system is configured to present a message indicating that the maximum number of measurements has been reached for a view and no additional measurements will be associated with the active view.

FIG. 9 is a diagrammatic view of a touch screen 160 with various functionalities for protocol creation with the diagnostic imaging system according to one aspect of the present disclosure. The functionalities for protocol record can include, but are not limited to, pause record, timer, print screen, alternate print, timer, end record, print view with view designation, e.g., view 4), view status, trim left, trim right, advance measures, and cine play. A present view selector includes previous view and next view selections, and displays an indicator for the present selected view (e.g., 4). In addition a cine speed selector includes decrease and increase selections, and displays an indicator for the present selected cine speed (e.g., 1).

In one aspect, the keys (or buttons) available on the protocol touch screen 160 during a protocol record may include the following. The Pause Record key is configured to pause the creation of protocol views, wherein no system actions are recorded in the current protocol and the state of the active view will be preserved for when the user returns from the paused state. The Print/Capture key is configured to trigger a Print/Capture, wherein the system is further configured to assign the appropriate image length (frame, number of seconds/beats, etc) to the active view, saves off all other view parameters (annotations, measurements, body markers), closes the active view and then creates a new view. The Timer key is configured to starts a timer. The View Status key is configured to toggle the display of the PLR. The End Record key is configured to conclude recording of the protocol, wherein the system simply stops recording the protocol. Upon selection of the End Record key, the protocol will be considered complete, and the protocol tab is removed from display. The system thereafter remains in the current system state and imaging mode, enabling the user to continue on with the exam. The View (rotary knob) is configured to allow a user to navigate back to previously recorded views. Other keys include the End. Exam key which is configured for ending the exam, wherein the End Exam button also conclude the recording of the protocol.

FIG. 10 is a diagrammatic view of one example of a protocol label region (PLR) view list 170 for protocol creation with the diagnostic imaging system according to one aspect of the present disclosure. The view list 170 includes a listing of views for the new protocol. As illustrated, views 1, 2 and 3 are completed, as indicated by the presence of a check-mark in the box next to the corresponding view identifier. View 4, however, is not yet completed, as indicated by the absence of a check-mark in the box next to the corresponding view identifier.

In one aspect, the PLR is configured to show the status of the protocol being created (170 of FIG. 10). Completed views include a “check mark” or other suitable indicator beside their corresponding label in the main view list portion of the protocol. The current active view is highlighted in the same manner as an active view on a normal protocol, a rectangle surrounding the selected view. The current active view being created will also be colored in analysis orange) in order to identify it to the user as the Active Recording View, ARV. If the user rotates the view knob to a previously completed view, the selected view will be highlighted in a given manner (e.g., rectangle surrounding the selected view). In one aspect, the ARV is colored as “analysis orange”.

FIG. 11 is a diagrammatic view of one example of a protocol label region active view region 180 for protocol creation with the diagnostic imaging system according to one aspect of the present disclosure. The active view region 180 includes an identification of a mode (e.g., 2D) for view 4 and measurements taken with respect to the content of view 4.

For example, the measurements shown include Spleen L of 7.79 cm and Spleen H of 8.99 cm.

In one aspect, the lower portion of the PLR (180 of FIG. 11) shows the detailed active view information and includes the name of the view in the header bar. Below the view name system imaging mode and measurement information. System imaging mode information reflects the current system imaging mode, wherein particular mode information requirements are dependent on implementation of the AIMS, Automatic Imaging Mode Switching feature. Any measurements completed since the creation of the active view will have the measurement label and result displayed.

With respect to capturing images while recording a new protocol, the view name is displayed on the Print/Capture touchscreen key. The protocol record functionality is configured to recognize any type of capture on the system, including prints, loops, cine captures, 3D freehand, 3D motorized, 3D STIC and 4D. While in 3D mode, the protocol recorder is further configured to be able to distinguish between volumes captured while in a 3D mode, loops captured while in a 3D mode, and prints captured while in a 3D mode. With respect to view annotations while recording new protocol, any annotations entered into the system are recorded. The position of the annotation is also recorded. If an annotation is positioned on the lower right corner of the display for one view, and the upper left corner of the display for another, those positions will be maintained when executing the protocol. The state of the annotation when the view is completed (via a capture) determines the final definition for the view. Annotations remain in place when the recorder creates a new view. Protocols will only modify a currently displayed annotation if the user selects a previously completed view that launches an associated annotation for that view.

With respect to body markers, any body markers, and their respective position(s), activated during a view are recorded in the protocol, along with the scan plane marker. The state of the body marker when the view is completed (via a capture) determines the final definition for the view. A body marker remains in place when the recorder creates anew view. Protocols modify a currently displayed body marker only if the user selects a previously completed view that launches an associated body marker for that view.

With respect to measurements, all measurements taken during view are recorded in the protocol. The measurement is associated to the view once the user selects a label from the CLR. If the user does not label a measurement, (i.e., the measurement is left labeled as ‘Dist’), then the measurement is not recorded with the view. If the user selects a label and then selects ‘Erase Calipers’ before finishing the measurement, then the measurement is removed from the view. If a measurement was completed, and the user selects ‘Delete Last’, then the measurement is removed from the view. If the user completes the view by virtue of a print or capture and then deletes the measurement or erases the calipers, the measurement remains associated with the view.

With respect to imaging mode switches, upon completion of each view, the current imaging state is recorded for the view, as discussed herein with reference to Automatic Imaging Mode Switching.

FIG. 12 is a flow diagram view illustrating a method 190 for protocol creation with a diagnostic imaging system according to another aspect of the present disclosure. The method begins at 192 with selection of the “New Protocol” button from the protocol selection list. At step 194, the system enters a protocol standby state, in anticipation of a subsequent user selection. If the user selects a “Body Marker” option 196, then the method proceeds with adding a body marker to the view at step 198. The method then returns to step 194. If the user enters “annotations” at step 200, then the method proceeds with adding annotations to the view at step 202. The method then returns to step 194. If the user completes a “Measurement” at step 204, then the method proceeds with adding the measurement to the view at step 206 and also displaying the measurement in the protocol label region (PLR). The method then returns to step 194. If the user selection “Captures/Prints” an image at step 208, then the method proceeds with adding the capture type and system mode to the view at step 210. The method then proceeds to step 212, wherein view parameters are stored to the view, and the system creates a next new view, with no mode changes occurring when creating a new view. The method then returns to step 194 and repeats until the user exits the protocol recording feature, for example, by selecting the end record button, for example, as found on the touch screen 160 of FIG. 9. Accordingly, there has thus been presented herein a method of exam protocol creation in a diagnostic imagine system. The method comprises initiating a non-protocol diagnostic imaging exam with the diagnostic imaging system. Concurrently with implementation of the initiated non-protocol diagnostic imaging exam, the method includes recording procedural characteristics of the non-protocol diagnostic imaging exam, wherein recording includes automatic system recording. In addition, the method includes defining a diagnostic imaging exam protocol in response to the recorded procedural characteristics of the non-protocol diagnostic imaging exam, wherein defining includes producing an automatic system generated definition of the diagnostic imaging exam protocol as a function of information extracted from the recorded procedural characteristics. Defining can further include storing protocol definitions of the procedural characteristics into the protocol definition as the procedural characteristics of the non-protocol diagnostic imaging exam occur. Furthermore, the diagnostic imaging system can comprise one of an ultrasound diagnostic imaging system, a computed tomography diagnostic imaging system, a magnetic resonance diagnostic imaging system, and an x-ray diagnostic imaging system. In one aspect, the non-protocol diagnostic imaging exam includes a portion of a previously established protocol diagnostic imaging exam.

In another aspect, procedural characteristics include at least one selected from the group consisting of imaging mode settings used in acquiring an image view, annotations made to the acquired image view, body markers added to the acquired image view, and measurements taken with respect to contents of the acquired image view. In a further aspect, the procedural characteristics include elements of the non-protocol exam sufficient to provide a predetermined minimal definition of an exam protocol.

In yet another aspect, defining further includes storing the defined diagnostic imaging exam protocol as a set of instructions on computer readable media that are executable by a computer. In another aspect, the system generated definition of the defined diagnostic imaging exam protocol comprises computer executable instructions for acquiring a number of image views and corresponding image view elements, according to the procedural characteristics of the non-protocol diagnostic imaging exam.

In another aspect, the number to image views of the defined diagnostic imaging exam protocol is determined from a number of image views acquired during the non-protocol diagnostic imaging exam upon which the defined diagnostic imaging exam protocol is based. In addition, the defined diagnostic imaging exam protocol can comprise diagnostic imaging system commands configured to implement a given imaging mode and imaging mode settings for one or more of the image views to be acquired. Furthermore, the diagnostic imaging exam protocol further comprises imaging system commands configured to implement one or more different types of exams.

According to further aspects, recording can comprise non-user selectable recording and defining can comprise user selectable defining. Non-user selectable recording refers to the diagnostic imaging system being configured to automatically record the non-protocol diagnostic imaging exam. In addition, user selectable defining refers to the system user being able to select, via an appropriate input or keystroke on the diagnostic imaging system control panel, the start of an automated system generated definition of the diagnostic imaging protocol as a function of information extracted from the procedural characteristics recorded during the non-protocol diagnostic imaging exam. According to yet another aspect, the method includes recording wherein recording comprises user selectable recording. In the later instance, recording is enabled in response to a user selection of recording.

In a still further aspect, the method includes defining wherein defining comprises user selectable defining. In the later instance, defining is enabled in response to a user selection of the defining. User selectable defining further includes selecting a portion of the non-protocol diagnostic imaging exam in response to user input, still further wherein the automatic system generated definition of the diagnostic imaging exam protocol includes generating the definition based on only that portion of the non-protocol diagnostic imaging exam selected by the user.

In another aspect, the method further comprises displaying a status of the defined diagnostic imaging exam protocol on a display as the diagnostic imaging exam is being defined. Displaying further includes providing display highlights of an acquired image view and displaying elements of the image view having been defined, based on one or more user inputs and keystrokes to the diagnostic imaging system.

In another aspect, defining includes assigning pre-defined names to image views as they are acquired and displaying the corresponding names in a sequential order on the display. In the later instance, the method further comprises changing one or more of the pre-defined names assigned to the image views in response to user input. The method can still further comprise verifying one selected from the group consisting of (I) element definitions of an acquired image view and (ii) characteristics of the defined diagnostic imaging exam protocol.

In another aspect, wherein subsequent to recording and prior to defining, the method further comprises deleting select ones of a number of image views captured during the non-protocol diagnostic imaging exam in response to one or more user inputs and, keystrokes.

According to another aspect of the method of the present disclosure, the defined diagnostic imaging exam protocol further comprises instruction prompts for implementing procedural characteristic actions associated with a corresponding image view to be acquired. The instruction prompts include prompts for at least one or more of the following consisting of (i) acquiring an image, (ii) annotating an acquired image, (iii) placing body marker graphics on an acquired image, (iv) performing named measurements with respect to content of an acquired image, and (v) terminating an exam. In one aspect, the method further comprises replaying the defined diagnostic imaging exam protocol for implementing a subsequent exam, wherein replaying includes at least one of (i) communicating the instruction prompts to a system user via the display and (ii) automatically performing one or more of annotating the acquired image, placing body marker graphics on the acquired image, and performing named measurements with respect to content of the acquired image.

Although only a few exemplary aspects have been described in detail above, those skilled in the art will readily appreciate that many modifications are possible in the exemplary aspects without materially departing from the novel teachings and advantages of the aspects of the present disclosure. For example, the aspects of the present disclosure can be applied to any diagnostic imaging system or medical imaging modality in addition to Ultrasound, such as Computed Tomography, Magnetic Resonance Imaging, X-Ray, etc. Accordingly, all such modifications are intended to be included within the scope of the aspects of the present disclosure as defined in the following claims. In the claims, means-plus-function clauses are intended to cover the structures described herein as performing the recited function and not only structural equivalents, but also equivalent structures. In addition, any reference signs placed in parentheses in one or more claims shall not be construed as limiting the claims. The word “comprising” and “comprises,” and the like, does not exclude the presence of elements or steps other than those listed in any claim or the specification as a whole. The singular reference of an element does not exclude the plural references of such elements and vice-versa. One or more of the aspects may be implemented by means of hardware comprising several distinct elements, and/or by means of a suitably programmed computer. In a device claim enumerating several means, several of these means may be embodied by one and the same item of hardware. The mere fact that certain measures are recited in mutually different dependent claims does not indicate that a combination of these measures cannot be used to an advantage.

1. A method of exam protocol creation in a diagnostic imaging system comprising:
initiating a non-protocol diagnostic imaging exam with the diagnostic imaging system;
recording, concurrently with implementation of the initiated non-protocol diagnostic imaging exam, procedural characteristics of the non-protocol diagnostic imaging exam, wherein recording includes automatic system recording; and
defining a diagnostic imaging exam protocol in response to the recorded procedural characteristics of the non-protocol diagnostic imaging exam, wherein defining includes producing an automatic system generated definition of the diagnostic imaging exam protocol as a function of information extracted from the recorded procedural characteristics.

2. The method of claim 1, wherein procedural characteristics include at least one selected from the group consisting of imaging mode settings used in acquiring an image view, annotations made to the acquired image view, body markers added to the acquired image view, and measurements taken with respect to contents of the acquired image view.

3. The method of claim 1, wherein defining further includes storing the defined diagnostic imaging exam protocol as a set of instructions on computer readable media that are executable by a computer.

4. The method of claim 1, wherein defining further includes storing protocol definitions of the procedural characteristics into the protocol definition as the procedural characteristics of the non-protocol diagnostic imaging exam occur.

5. The method of claim 1, wherein the system generated definition of the defined diagnostic imaging exam protocol comprises computer executable instructions for acquiring a number of image views and corresponding image view elements, according to the procedural characteristics of the non-protocol diagnostic imaging exam.

6. The method of claim 1, wherein a number of image views of the defined diagnostic imaging exam protocol is determined from a number of image views acquired during the non-protocol diagnostic imaging exam upon which the defined diagnostic imaging exam protocol is based.

7. The method of claim 6, wherein the diagnostic imaging exam protocol further comprises imaging system commands configured to implement one or more different types of exams.

8. The method of claim 1, wherein recording is non-user selectable and defining is user selectable.

9. The method of claim 1, wherein defining further comprises user selectable defining, wherein defining is enabled in response to a user selection of the defining.

10. The method of claim 1, wherein the procedural characteristics include elements of the non-protocol exam sufficient to provide a predetermined minimal definition of an exam protocol.

11. The method of claim 1, further comprising:
displaying a status of the defined diagnostic imaging exam protocol on a display as the diagnostic imaging exam is being defined.

12. The method of claim 1, wherein defining includes assigning pre-defined names to image views as they are acquired and displaying the corresponding names in a sequential order on the display.

13. The method of claim 1, wherein subsequent to recording and prior to defining, the method further comprising:
deleting select ones of a number of image views captured during the non-protocol diagnostic imaging exam in response to one or more user inputs and keystrokes.

14. The method of claim 1, wherein the defined diagnostic imaging exam protocol further comprises instruction prompts for implementing procedural characteristic actions associated with a corresponding image view to be acquired.

15. The method of claim 14, further wherein the instruction prompts include prompts for at least one selected from the group consisting of acquiring an image, annotating an acquired image, placing body marker graphics on an acquired image, performing named measurements with respect to content of an acquired image, and terminating an exam.
